(19) United States
(12) Patent Application Publication
(10) Pub. No.: US 2002/0133108 A1
(43) Pub. Date: Sep. 19, 2002

(54) DEVICE FOR STABILIZING A JOINT

(76) Inventor: MICHAEL JAGODZINSKI, HEIDELBERG (DE)

Correspondence Address: NIXON & VANDERHYE, 1100 NORTH GLEBE ROAD, 8TH FLOOR, ARLINGTON, VA, 222014714

(21) Appl. No.: 09/423,829

(22) PCT Filed: Mar. 18, 1999
(86) PCT No.: PCT/EP99/01815

(30) Foreign Application Priority Data
Mar. 19, 1998 (DE) 198 11 925.9

(51) Int. Cl. A61F 2/72

(52) U.S. Cl. 602/16; 623/26

(57) ABSTRACT

A device for stabilizing a joint, more particularly for a knee joint, comprising a first fixing splint (3), a second fixing splint (5) and a movement control mechanism (7 and 9) bridging over the joint and having flexible connector (8), which to the sides of the joint connects the first fixing splint (3) with the second fixing splint (5). The flexible connector (8) extends from one front side of the first fixing splint (3) on either side past the joint to a rear region of the second fixing splint (3) in order to bias the first fixing splint toward the joint and to the rear and to bias the second fixing splint (5) toward the joint and to the fore.

The invention relates to a device for stabilizing a joint, more particularly a knee joint, comprising a first fixing splint, a second fixing splint and a movement control mechanism bridging over the joint and having a flexible connector, which on the sides of the joint connects the first fixing splint with the second fixing splint

Knee orthotic devices are known having a thigh and a lower leg splint, which using plastic tapes with burr fasteners are respectively able to be applied on the thigh and, respectively, the lower leg and which are connected with a hinge joint for bridging over the knee joint. The hinge joint between the thigh splint and the lower leg splint has a fixed pivot, about which the thigh and the lower leg splint are able to be pivoted in relation to one another. Using metal pins or plastic elements it is possible for the pivotal movement to be limited to between 0? and 140?.

In the case of known knee orthotic devices there is the disadvantage that the human knee joint can not be completely imitated and stabilized by knee orthotic device with a hinge joint. Thus it has been shown in clinical studies that in the case of present day conventional knee orthotic devices there is merely a reduction of tension in the cross ligament. Furthermore circular or all-round fixation of the thigh and lower leg splint on the front muscles of the thigh and lower leg leads to interference with the circulation of the blood, for instance in the muscles of the calf, and on the other hand to disadvantageous changes in gait.

The European patent publication 0 432 303 A1 discloses a device for stabilizing a knee joint comprising a thigh fixing splint and a lower leg fixing splint, which are connected together in an articulating fashion by means of two lateral arms. These two fixing splints are able to be set in position by means of adjustable cuffs on the thigh and on the lower leg. On one side of this known device a flexible connector is provided, which causes the cuffs to be drawn tighter around the thigh and the lower leg respectively on extension flexure of the leg.

In connection with cross ligament tears there is also the problem that the relief from tension of the cross ligaments in the direction of the injured cross ligament in the extreme positions must be particularly extensive, whereas the relief from tension in the direction of the torn cross ligament in the flexed position may be less. In the case of the knee orthotic device in accordance with the said European patent publication 0 432 303 A1 there is the disadvantage that although the knee orthotic device is applied to the leg with a different degree of firmness in a manner dependent on the position of the thigh, there is however no individually adapted stabilizing effect of the orthotic device, which is able to replace the proper function of the cross ligament by a higher tension force in the direction thereof, more especially in the extreme positions of the leg.

One object of the invention is to provide a device for stabilizing a joint of the type mentioned, which provides for an improved stabilization of the knee joint and in this respect brings about an additional reduction of tension at the ligaments of the joint to be stabilized.

This object is attained in accordance with the invention by the features of claim 1. In accordance therewith the tension member extends from one front side of the first fixing splint on either side past the joint to a rear region of t second fixing splint in order to bias the first fixing sp t toward the joint and to the rear and to bias the second fixing splint toward the joint and to the fore.

The invention is based on the recognition that stability of a joint following a ligament tear, more particularly a cross ligament rupture, can be advantageously achieved by a flexible connector, which imitates the front and/or rear cross ligament clear of the knee joint. A particular advantage in this respect is that the stabilizing effect and gait function of the joint of the patient after putting on the knee orthotic device during flexure and extension is practically the same as is the case with an uninjured cross ligament. The thigh splint applied to the thigh and the lower leg splint applied to the lower leg are so bound by the flexible connector to the sides of the joint that the flexible connector is pivoted on the fixing splints just where the extra-articular projection of the isometric point, for instance of the front cross ligament, is located. This means that the flexible connector is arranged on the outer side of the knee joint and possesses a course corresponding to the cross ligaments in the knee, it only being an imitation clear of the knee joint.

The knee orthotic device does in this respect more particularly prevent a front instability in the extended positions of the knee joint, since this is in accordance with the principal effect of the front cross ligament imitated by the flexible connector. The knee orthotic device of the invention in this respect functions to counteract instability during walking and more especially in the extension phase of the stabilized knee joint. The longitudinal tension on transplanted tissue increases more particularly during extension of the knee joint, for which reason the use of a knee orthotic device in accordance with the invention having cross ligaments externally imitated by the flexible connector must be regarded as being particularly advantageous.

A distinct advantage of the knee orthotic device of the invention is accordingly to be seen in the arrangement of the flexible connector, which exerts a tension force dependent on the state of flexure of the knee joint on the lower leg toward the thigh. If the front cross ligament is non-functional it is possible for the tension direction to be to the rear on the shin, the maximum tension effect being at a flexure of 30?. In the case of non-functionality of the rear cross ligament the tension direction of the lateral flexible connector between the lower leg fixing splint and the thigh fixing splint is so set up that the maximum tension is reached at 70? flexure of the knee joint, the flexible connector being arranged laterally on the knee as seen in side view looking toward the rear cross ligament and maximum tension is attained at 70? flexure of the knee joint.

The flexible connector is preferably designed in the form of a cable or flexible plastic or wire rod. As a cable it is for instance possible to provide a piece of plastic cable, which is arranged on either side of the knee joint, or it is possible to provide several pieces of cable separately, which connect the first fixing splint with the second fixing splint at the sides of the joint. Instead of a cable it is however also possible to provide a flexible plastic or wire rod, which then for example provides increased resistance on displacement of the two fixing splints about a pivot. The flexible connector is only to imitate the cross ligaments extra-articularly and for this reason imitate the properties thereof as regards taking up tension forces in an optimum fashion. In this respect it is possible to provide for the flexible connector to have elastic properties to a certain extent.

In the case of a first further development of the invention the flexible connector extends from the front side of the first fixing splint, for example the lower leg fixing splint, to the side past the joint to a rear region of the lateral face or to the rear side of the second fixing splint, for instance the thigh fixing splint. It is pointed out that the device for stabilizing a joint is not to be limited to knee orthotic devices. The device of the invention may also be utilized for stabilizing all other joints of living creatures. It is only for simplification that the device for stabilizing a joint in the form of a knee orthotic device is shown. To the extent that in the following passages there is a mention of the front side of a joint, this is to be understood to be a reference to the side, on which the joint movement is halted so that the joint is not able to be moved in the forward direction any further. In the case of the knee joint the front side of the joint is consequently the side to the fore in the direction of stepping.

In the simplest case two flexible connectors are provided, which respectively extend along the sides of the joint and connect the thigh splint with the lower leg splint. During walking along the knee is stabilized because the lower leg splint is biased underneath knee joint to the rear, whereas the thigh splint is biased by the flexible connector forward above the knee joint.

It is preferred for the flexible connector on the side face of the second fixing splint and more particularly of the thigh splint, to be able to be redirected on a redirecting support and to extend thence to the front side of the second fixing splint. The flexible connector is in this respect on the one hand secured on the front side of the lower leg splint and on the other hand on the front side of the thigh splint. Accordingly the redirection of the cable on the one hand causes the thigh splint to be biased or loaded toward the front side of the thigh and on the other hand attachment to the front on the thigh splint means that the flexible connector is accessible on the front side, for example for resetting or opening, as for example is necessary for putting on the device.

In the case of a particularly preferred further development of the device in accordance with the invention the flexible connector is designed in the form of a cable ring. The cable ring in this case comprises a plastic cord, which is able to be connected or disconnected by a catch or closure as for example a snap-action catch or detent for the cable ring. In accordance with the invention the cable ring can be secured on the front side at the top on the lower leg splint and thence extend on either side of the knee joint to the rear in order at this position to be able to be redirected or bent at the point of redirection adjacent to rear side face of the thigh splint in a forward direction. From the lateral redirecting supports on the thigh splint the cable ring extends toward the front face of the thigh splint, as for example to a cable ring catch or lock. The cable ring catch permits an opening of the cable ring in order to put on and take off the knee orthotic device.

It is preferred for a first setting element to be provided for shifting the attachment or redirection point for the flexible connector on the front side of the first fixing splint, i.e. the lower leg splint. The setting means may for instance comprise a plurality of attachment point means arranged like a grid, for a cable ring with the result that same may be hooked and attached at different attachment positions and may then be biased in a direction away from knee joint. It may however be a question of any other desired form of a tensioning device for rendering possible an optimum positioning of the cable in relation to the first fixing splint.

Furthermore a cable shortening means may be additionally employed for setting the length of the flexible connector. It is preferred for the cable shortening means to be arranged on the front side of the thigh splint in order to render possible simple operation by the patient. Thus it is possible for the cable shortening means to be a pulley adapted to rotate about an axis of rotation, turning of the pulley winding up the cable ring about the axis of rotation so that the flexible connector is shortened. This means that the device for stabilizing a joint may be adapted to different patients in an optimum fashion and using the cable shortening means a predetermined biasing of the device, i.e. as affecting the relative position of the thigh and lower leg splints, may be set so that the stabilizing function is for example also able to be adapted to the weight of a patient. By means of the cable it is accordingly possible to produce an adaptation of the device to suit different sizes of joint, something which permits a particularly high degree of flexibility when using the knee orthotic device,

In accordance with a preferred development of the invention the thigh and also the lower leg are engaged at semicircular contact zones. Unlike the case of known knee orthotic devices, which completely surround the thigh and the lower leg muscles and in the case of which it is necessary to firmly engage the thigh and the lower leg in order to render possible any stabilizing effect, in the case of the device of the invention the muscles are not now constricted. With the flexible connector in accordance with the invention the splint is biased forward and toward the knee joint and the lower leg splint is biased upward toward the knee joint. Consequently the first fixing splint, more particularly the lower leg splint, is only closed at the front side of the lower leg, whereas the rear side of the lower leg may be left uncovered. The lower leg splint is preferably molded on the lower leg, is provided with padding means and may be extended in a rearward direction at the sides of the lower leg to provide an improved support function. Since however no circular encircling action is required for the lower leg, neither the blood vessels generally nor the muscles are constricted by plastic tapes with a burr fastening extending in a ring around the lower leg.

It is possible for the second fixing splint, and more especially the thigh splint, to be open at the front side. In accordance with the invention the thigh splint possesses a rear support wall and two lateral support walls, which are able to be connected with each other. It is preferred for two redirecting point means to be provided in the rear region at the lateral walls. In the front region of the lateral support faces to the left and to the right a respective support or bearing-means for the forwardly extending flexible connectors are provided, which extends over the exposed thigh to the front on the open side of the thigh splint, where preferably a cable shortening means is provided. It is possible naturally to provide padding on the open side of the thigh splint for the protection of the exposed thigh against injury by the cable shortening means.

The redirection of the flexible connector at the two lateral redirection point means that in the case of redirection of the first fixing splint, for example in the forward direction, the two lateral support walls of the second fixing splint, more particularly of the thigh splint, are moved inward toward each other. In this case the support of the flexible connector produces forces at the front side of the lateral support walls with the result that the support walls are more intensely thrust against the thigh.

It is preferred for the fixing splints and more particularly however the thigh splint, to be adjustable as regards the periphery thereof. For instance it is possible for the thigh splint to comprise three separate splint walls, namely a rear wall and two lateral walls. The rear wall can possess an adjusting mechanism, as for example a groove and tongue connection, using which the side walls are able to be secured at different distances apart on the rear wall. Accordingly the thigh splint may be set to different peripheral sizes of thigh.

The two side faces are in this case preferably made to correspond to the form of the femur adjacent to the knee joint. For instance it is possible for the thigh splint to taper from the top to the bottom, this preventing any slipping of the thigh splint, and more particularly of its side walls, over the knee joint.

Using an elastic flexible connector it is possible to ensure a certain degree of freedom of motion in the knee joint to stabilized. This may be an advantage as regards the gait possible with the knee orthotic device employed.

A fixing splint, and more particularly a lower leg splint, may possess a holding means, which counteracts the tension forces of the flexible connector acting toward the joint. Such holding means is preferably an encircling heel strap. Using the holding means it is possible for the lower leg splint to be additionally biased toward the foot with the result that the tension forces of the device toward the knee are even better able to be equalized. The tension forces of the device in accordance with the invention accordingly increase the angle of the thigh in relation to the vertical and consequently stabilize the knee joint additionally in the case of an increased angular displacement of the knee. It is more particularly in the case of use of the device of the invention for a rehabilitation phase with sport, that the stabilizing effect on the knee joint may be additionally increased by the hell encircling strap and by an increase in the tension forces on the flexible connector.

In accordance with an advantageous form of the device the movement control mechanism comprises a guide means for the displacement along a curve of the support point means of a fixing splint. The two fixing splints may thus be connected also via a rotary bearing means, which on the one hand permits pivoting of the two fixing splint and on the other hand a displacement of the pivot along a predetermined curve. The displacement curve of the pivot may be adapted to the natural joint motion and also have a tensioning means, as for example a spring, whose biasing action is preferably toward the joint and to the rear.

The displacement in accordance with the invention of the pivot takes into account that the flexure of the knee joint is not simply able to be imitated by a hinge with a fixed pivot. The pivot between the two fixing splints is displaced in accordance with the invention along the displacement curve corresponding to the anatomy of the limb. In accordance with the invention there is also a provision such that the movement control mechanism for displacement of the pivot along a curve is able to be combined with the movement control mechanism having the flexible connector. It is in this manner that a particularly advantageous device for the stabilization of a joint is produced.

Preferably the curved guide means possesses a guide groove, a guide rail or a special-purpose pivot bearing, which renders possible the above mentioned equalization movement of the two fixing splints. In the simplest case one fixing splint, more particularly the lower leg splint, has two laterally arranged pins, which respectively fit into laterally arranged curved guide grooves in the movement control mechanism or on the thigh splint. The pins with the lower leg splint secured thereto may consequently be shifted along the curved guide groove in such a fashion that the pivot between the fixing splints, which is defined by the pins, is able to be shifted following a curve.

Further advantageous embodiments of the invention will be gathered from the dependent claims and the description. A plurality of embodiments of the invention are illustrated in the diagrammatic drawing and will be described in the following.

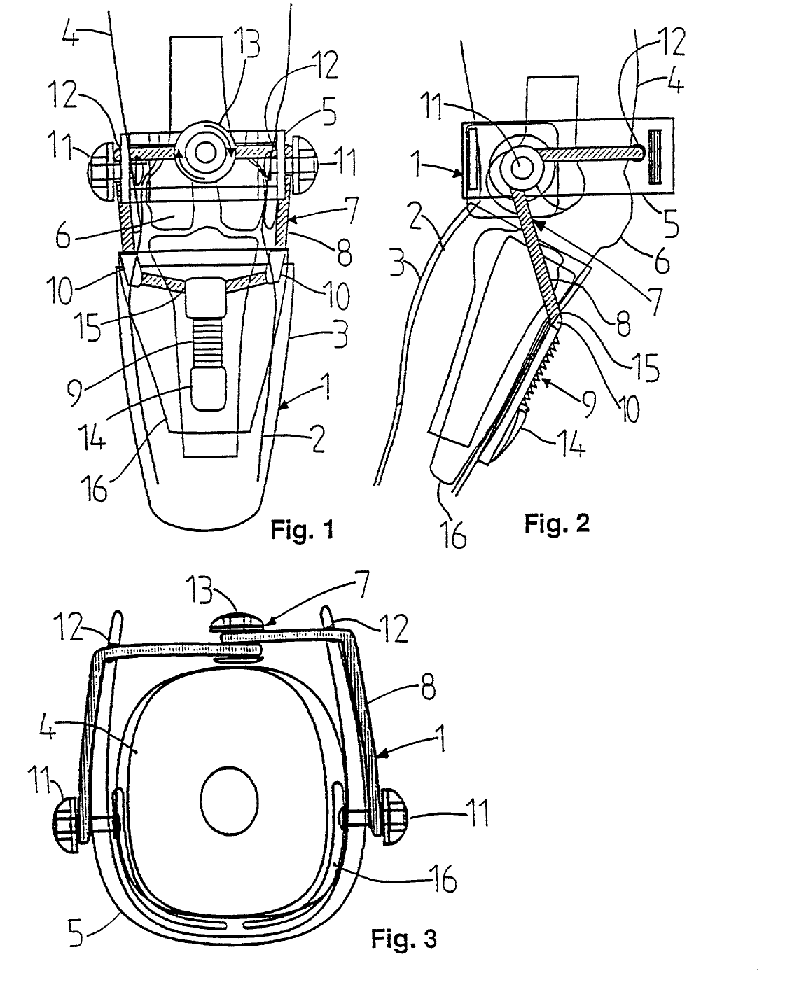

FIG. 1 is a front elevation of the device in accordance with the invention as a first embodiment thereof.

FIG. 2 is a side view of the device in accordance with the invention as the first embodiment thereof.

FIG. 3 is a plan view of the device of the invention in accordance with the first embodiment.

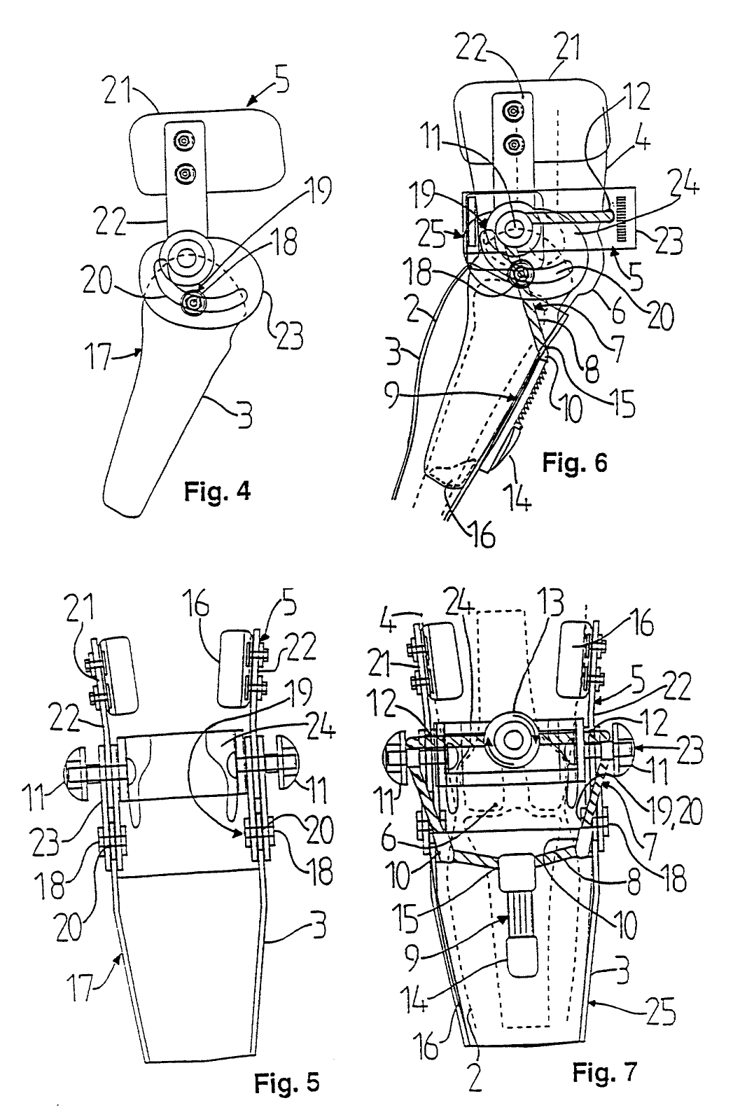

FIG. 4 is a lateral view of a part of the device in accordance with the invention according to a second embodiment.

FIG. 5 shows a front view of the part of the device of the invention as in FIG. 4.

FIG. 6 is of a device in accordance with the invention as applied to a leg in accordance with the second embodiment.

FIG. 7 is a front elevation of the device applied to the leg according to the second embodiment.

The knee orthotic device 1 in accordance with the invention possess a lower leg splint 3 adapted to be secured to the lower leg 2, a thigh splint 5 adapted to be applied to the thigh 4 and a movement control mechanism 7 bridging over the knee joint 6. The lower leg splint 3 is adapted to be arranged underneath the knee joint 6 and the thigh splint 5 above the knee joint 6 and they are connected together with the aid of the movement control mechanism 7.

The movement control mechanism 7 comprises a cable 8, which forms a closed or complete ring or loop. The cable 8 is attached to the an adjustment element 9 in a top region of the lower leg splint 3. The cable 8 is in addition mounted, so that it can be shifted, by means of two fixing elements 10 arranged on the lower leg splint and extends on either side of the knee joint respectively to a redirecting means 11 mounted on the thigh splint 5 to the side and to the rear. The cable 8 extends at the sides of the knee joint 6 from the front side of the lower leg 2 to the rear side of the thigh 4. This means that the knee orthotic device 1 in accordance with the invention produces forces corresponding to the natural tension forces of the human cross ligament at the knee joint 6, outside the leg.

From the two redirecting means 11 the cable 8 extends along the side walls of the thigh splint 5 forwardly and is guided by means of further guide element means 12, as for example holes in the side walls, on the front side of the thigh splint 5 to a cable shortening means 13.

The cable shortening means 13 is a rotary pulley, whose operations causes shortening or lengthening of the length of the cable 8. This means that the tension produced by the cable 8 between the thigh splint 5 and the lower leg splint may be altered. The side walls of the thigh splint 5 may be provided with a padding means, as for instance an air-filled cushion, adapted to the shape of the femur.

The setting element 9 causes a displacement of the bottom attachment point of the cable 8 on the lower leg splint 3. By actuation of the setting element it is possible for the attachment point means to be reset in position along the front side of the lower leg splint 3 in a direction parallel to the run of the lower leg. The setting element 9 is designed like a toggle-action buckle 14 and may shift the bottom point means 15 of the cable 8 through a predetermined distance. This means that the cable 8 is tensioned in a downward direction. Furthermore the design may be such that for taking off the knee orthotic device 1 the cable 8 is able to be detached from the bottom attachment point 15. As an alternative it is possible for the cable 8 to be disconnected adjacent to the cable shortening means 13 at a detent catch.

The course of the cable 8 in accordance with the invention between the front rear side of the lower leg splint 3 and the rear side of the thigh splint 5 is responsible for stabilization of the knee joint 6 by drawing the lower leg splint 3 backward and by drawing the thigh splint 5 forward. The cable 8 could for this reason also be completed as a loop on the rear side of the thigh splint 5 without redirection at redirection point means 11.

The lower leg splint 3 comprises a splint molded to fit the lower leg 2 and having a padded lining. The lower leg splint 3 is not closed at the rear side of the lower leg 2 and consequently has an essentially semicircular cross section. The thigh splint 5 also has a semicircular cross section and is closed at the rear side of the thigh 4 and at the front side of the thigh 4 it is open. In this case as well padding 16 serves to improve the comfort for the user in the thigh splint 5. The lower leg splint 3 a splint which is elongated in the direction along the lower leg 2, whereas the thigh splint 5 constitutes a narrow semicircular stiff band or tape segment of plastic fitting around the thigh 4. The thigh splint 5 may consist of a separate rear wall and side walls adapted to be mounted thereon in an adjustable fashion.

In FIGS. 4 and 5 a part of a second embodiment of the present invention will be seen. In this case identical or similar elements have the same reference numerals as used for the first embodiment. The knee orthotic device 17 has a lower leg splint 3 with two laterally arranged bearing pins 18, which are able to be reset along a curved guide groove 20 arranged in the movement control mechanism 19. The guide groove 20 provided to the side on the movement control mechanism 19 opposite to the side walls of the lower leg splint 3 constitute, together with the bearing pins 18, a locally shiftable pivot between the movement control mechanism 19 and the lower leg splint 3. The thigh splint 5 is made in two parts, with a top part 21 having internal padding 16, which via two laterally arranged ribs 22 is connected with a bottom part 23 of the thigh splint.

The bottom part 23 of the thigh splint 5 has a receiving region 24 optimized in its shape for a bottom section of the thigh 4 of the patient. For combination with a knee orthotic device 1 with a cable 8 the knee orthotic device 17 has specially prepared lateral redirecting means 11. The part, illustrated in FIG. 4 and 5, of the second embodiment in accordance with the invention serves for stabilizing the knee joint. It is possible also to provide an elastic element, as for example a spiral spring, which urges the bearing pins 18 into a rear or front region of the guide groove preferentially.

As shown in FIGS. 6 and 7 the second embodiment of the knee orthotic device 25 in accordance with the invention entails having a combination of the first embodiment with the device illustrated in FIGS. 4 and 5. This leads to a knee orthotic device having an excellent stabilizing effect and which in addition has a pivot able to be moved along the curved guide groove 20 in an anatomically adapted manner. On the lower leg splint 3 a heel encircling strap may be provided, not illustrated, which holds the lower leg splint in its position and prevents displacement toward the knee joint.

1. A device for stabilizing a joint, more particularly for a knee joint, comprising a first fixing splint (3), a second fixing splint (5) and a movement control mechanism (7 and 19) bridging over the joint and a having flexible connector (8), which to the sides of the joint connects the first fixing splint (3) with the second fixing splint (5), characterized in that the flexible connector (8) extends from one front side of the first fixing splint (3) on either side past the joint to a rear region of the second fixing splint (3) in order to bias the first fixing splint toward the joint and to the rear and to bias the second fixing splint (5) toward the joint and to the fore.

2. The device as claimed in claim 1, characterized in that the flexible connector (8) is a cable or a plastic or wire rod.

3. The device as claimed in claim 1 or in claim 2, characterized in that the flexible connector (8) extends from the front side of the first fixing splint (3) to a rear region of the side face or to the rear side of the second fixing splint (5).

4. The device as claimed in any one of the claims 1 through 3, characterized in that the flexible connector (8) is able to be redirected at the side face of the second fixing splint (5) by means of a redirecting support (11) and thence extends to the front side of the second fixing splint (5).

5. The device as claimed in any one of the claims 1 through 4, characterized in that the flexible connector (8) is in the form of a cable ring.

6. The device as claimed in any one of the claims 1 through 5, characterized in that a first setting element (9) is provided for resetting the position of attachment or the redirection point means for the flexible connector on the front side of the first fixing splint.

7. The device as claimed in any one of the claims 1 through 6, characterized in that the length of the flexible connector(8) is able to be adjusted by means of a cable shortening means (13).

8. The device as claimed in claim 7, characterized in that the cable shortening means (13) is arranged on the front side of the second fixing splint (5).

9. The device as claimed in any one of the claims 1 through 8, characterized in that with the device secured to the stretched joint the first fixing splint(3) is closed on the front side of the joint.

10. The device as claimed in any one of the claims 1 through 9, characterized in that the second fixing splint(5) is open on the front side.

11. The device as claimed in any one of the claims 1 through 10, characterized in that the second fixing splint (5) is able to be adjusted with respect to its periphery.

12. The device as claimed in any one of the claims 1 through 11, characterized in that the flexible connector (8) is elastic.

13. The device as claimed in any one of the claims 1 through 12, characterized in that the fixing splint possesses a holding means, which counteracts the tension forces of the flexible connector(8) acting toward the joint.

14. The device as claimed in claim 13, characterized in that the holding device comprises a heel encircling means.

15. The device as claimed in any one of the claims 1 through 14, characterized in that the movement control mechanism (19) comprises a guide means for displacement of the bearing point means of a fixing splint along a curve.

16. The device as claimed in claim 15, characterized in that the curved guide means comprises a guide groove (20), a guide rail or a special-purpose pivot.

17. The device as claimed in claim 15 or in claim 16, characterized in that a fixing splint comprises two laterally arranged pins (18), which respectively fit into a curved guide groove (20)laterally arranged respectively on the movement control mechanism (19).
